(19) United States
(12) Patent Application Publication
(10) Pub. No.: US 2021/0077024 A1
(43) Pub. Date: Mar. 18, 2021

(54) STABILIZATION OF EAR DEVICES

(71) Applicant: ZIPLINE HEALTH, INC., San Francisco, CA (US)

(72) Inventor: Ryan BOUCHER, San Francisco, CA (US)

(21) Appl. No.: 16/620,427

(22) PCT Filed: Jun. 8, 2018
(86) PCT No.: PCT/US2018/036715

§ 371 Date: Dec. 6, 2019

Related U.S. Application Data
(60) Provisional application No. 62/517,852, filed on Jun. 9, 2017.

(51) Int. Cl. A61B 5/00; A61B 5/01

(52) U.S. Cl. CPC A61B 5/6817; A61B 5/01; A61B 2560/04; A61B 2562/0271; A61B 2562/0233; A61B 2562/16

(57) ABSTRACT

Systems and methods are described herein for obtaining diagnostic data from a subject's ear. The device has an inserted position, a rotated position, a central body, an extension, and a receptor. The extension is connected to the central body and is adapted for insertion into the ear canal. In some implementations, the device comprises a first flexible arm and a second flexible arm extending from the central body. At least one of the flexible arms bends to confirm o the concha of the ear when the device is in the rotated position. The first and second flexible arms are configured to encourage a placement of the device such that the receptor is able to obtain the diagnostic data from the ear drum when the device is in the rotated position.

### CROSS REFERENCE TO RELATED APPLICATIONS

This application claims priority to U.S. provisional application No. 62/517,852, filed Jun. 9, 2017, the content of which is hereby incorporated herein by reference in its entirety.

### BACKGROUND

Subjects with ear injuries or undiagnosed ear pain are typically forced to visit one or more physicians or medical treatment centers to have their condition diagnosed. If the subject is a small child, or if the condition is thought to be serious or in need of immediate treatment, the long waiting periods for a physician appointment may seem unreasonable or unacceptable, and the subject often ends up going to an emergency room or urgent care center or both on a “walk-in” basis. At the medical facility, the subject (or his or her guardian or caregiver) fills out medical history forms, answers questions about the condition, and has a clinician perform a physical examination to learn about the condition. In many cases, the waiting period at the facility can consume many hours, depending upon facility capacity and other subject needs. Ultimately, the subject's visit may have been unnecessary, as the condition may not have truly been “urgent” or “critical.” and thus treatment could have been delayed and accommodated at a regularly scheduled appointment, or the condition would have resolved itself with little or no intervention by the physician. In many cases, the stress and uncertainty associated with the subject's condition can be more debilitating than the condition itself, especially where the subject is a small child, and the caregiver is an inexperienced parent who is anxious about his or her child's condition. Moreover, the unnecessary use of emergency and urgent care facilities levies a heavy cost on the nation's health care and health care insurance systems, as such services are generally much more expensive than similar services provided on a scheduled or appointed basis.

In particular, in many cases, ear ailments could be diagnosed using visual information obtained at a remote location. Obtaining visual data of a patient's ear, however, can be time-consuming, ineffectual, or even dangerous, for an inexperienced lay person due to the shape and anatomical structures of the patient's ear. Current ear diagnostic devices are typically difficult to use and usually require training and practice. Additionally concurrent ear diagnostic devices can create (such as, for example, if the patient is in distress and requires immediate medical attention) tolerability and safety problems, especially when in the hands of an inexperienced consumer rather than a trained professional. For example, in order to image the ear drum with an otoscope, several variables need to be controlled. One hand is used to pull the ear back relative to the head in order to straighten the ear canal. The other hand is used to hold and position the otoscope. In order to maintain the position of the ear and otoscope relative to the head, one hand is often braced against the head and usually also against the otoscope which is being held in the other hand. All of these variables, as well as the orientation and depth of the tip of the speculum (portion of the otoscope which is inserted into the ear canal), are adjusted and controlled by the user in order to safely use the device and image the ear drum. A user should be able to see or feel the position of these moving parts while looking through a lens or at screen to know if the ear drum is in view. This is a difficult enough operation to describe, let alone to perform, and usually requires training, practice and a certain degree of perception or “feel”. Additionally, caution must be used so as to avoid insertion of the speculum too far as well as to avoid pressure of the tip of the speculum against the walls of the ear canal.

### SUMMARY

Systems, methods and devices are described herein for obtaining visual data related to a subject's inner ear. The systems and devices described herein can be placed with little to no interference by anatomical features of the ear or surrounding features. Such system and devices can enable an untrained layperson to safely obtain accurate visual information relating to the subject's ear, which may then be transmitted to a physician or other medical professional for diagnosis of the subject's ear condition.

In some implementations, a device obtains diagnostic data from a subject's ear. As used herein, “subject” may refer to one or more individuals desiring or needing medical advice. A subject may be a group, e.g. exercise class or sport team. In some implementations, the device is a medical device. For example, the device may be configured to obtain visual data relating to the subject's ear drum to diagnose an ailment and/or the device may be an infrared thermometer configured to take the subject's temperature. The subject's ear may include the outer ear (also referred to as the auricle), the middle ear, the inner ear, or any anatomical feature or landmark of the ear.

In some implementations, the device has an inserted position and a rotated position. In some implementations, in the inserted configuration, the device may be inserted into the ear without rotation. In some implementations, the device may reside in the rotated configuration after the device is inserted into the subject's ear and is rotated between 45 degrees and 180 degrees along a plane approximating the sagittal plane.

In some implementations, the device may comprise a central body. For example, the central body, may be an oval-shaped three-dimensional structure, a rounded rectangular prism, an earbud, a headphone, or any suitable shape. The central body may be sized and shaped to fit within or partially within the auricle. The central body may be made of a hard plastic, a soft plastic, polyurethane, a stiff material, a flexible material, or any suitable material. The central body may be formed in a variety of ways. For example, the central body may be molded, 3-D printed, hand-shaped, or any suitable method.

In some implementations, the central body may be stabilized by the ear in the rotated position. Moving the device from the inserted position to the rotated position may “lock” the device in place. For example, in the rotated position, ear tissue may contact portions of the device and apply forces to the central body such that the central body is held in a fully-seated position within or partially within the auricle. In some implementations, stabilizing the central body in the rotated position may enable the device to be operated in a hands free manner. For example, a user may place the device in a subject's ear in the inserted configuration, rotate the device into the rotated position, then “step away” from the subject, leaving the device held in place without the external user applying any additional forces. In some implementations, at least one of an inferior portion and a posterior portion of the central body may be held in place by the antitragus and an anterior portion of the central body may be held in place by the tragus.

In some implementations, the device may comprise an extension connected to the central body. The extension may be adapted for insertion into the ear canal. For example, the extension may be sized and shaped to fit within the ear canal of a subject. In some implementations, the extension may be an attachment coupled to the central body. For example, the extension may be a removable attachment that can be replaced or adjusted for maintenance or different ear canal shapes and/or sizes. In some implementations, the extension used for a baby's ear may be smaller and/or more flexible than an extension used for an adult's ear. In some implementations, the extension may be integrally formed with the central body. For example, the central body and the extension may not be de-coupled without breaking the device. The central body and extension may be formed from a single smooth piece of outer material. For example, the central body may taper into the extension. Any of the extensions described herein may include bulb or ball joints, expandable tubes, light tubes, LED configurations, tapered speculums, Q-tip configurations and/or spring plunger extensions.

In some implementations, the device may comprise a receptor located within at least one of the central body and the extension. The receptor may be an image capturing element (e.g., a camera), a medical device (e.g., a thermometer), an infrared sensor, or any suitable element. In some implementations, the receptor may be an image capturing element configured to obtain visual data of the ear drum in the rotated configuration. For example, when the device is placed in the inserted position the receptor may not be able to “see” or access the ear drum. When the device is adjusted into the rotated position, the receptor may be able to obtain data from the ear drum. For example, if the receptor is within the tip of the extension, when the device is placed in the inserted configuration, the extension may only extend a short distance within the ear canal, but the extension may extend further into the ear canal in the rotated positon, such that the receptor can image the ear drum.

In some implementations, the receptor may comprise an accessing element and a capturing element. For example, an access elements may transfer diagnostic information to and from capturing elements and areas of the body. Access elements include inputs and outputs such as lenses that transmit light from a light source towards an object as well as lenses that collect light and channel it towards a video chip to capture the light. Access elements may include any means to access, collect and transfer diagnostic information between an area of the body and a capturing element. These may include open channels, reflective surfaces and mirrors, fiber optics, lenses, diaphragms and other means to collect and transfer energy including heat, sound, electricity, light, motion, and magnetic fields. Access elements may also be used for tissue or fluids of the body. Access elements may conform to an area of the body where the shape, texture or other characteristic is of interest. Capturing elements include elements which are used to create and/or capture diagnostic-related information. These may include source elements, such as light sources and pressure sources, as well as destination elements such as video chips. For example, an LED uses electricity to produce light which can be transmitted towards an ear drum. This light is then absorbed or reflected back towards a lenses which focuses the light onto a video chip which translates the information into electricity to be processed (stored, recorded, output on screen etc.). A capturing element may also be a user's or provider's eye or ear, for example, for gathering light or sound which is then processed by the brain. Pressure transducers may be used to capture force in order to reproduce a tactile feel which is similar to nerve receptors on fingers.

In some implementations, the receptor may comprise an element configured to process audio data. For example, the receptor may comprise a microphone, speaker, or any other suitable sensor configured to process audio data. In some implementations, the receptor may comprise an element configured to obtain visual data. For example, the receptor may comprise a camera or any other suitable sensor configured to obtain or process visual data.

In some implementations, the device may comprise a first flexible arm and a second flexible arm. Each of the first flexible arm and second flexible arm has a connected end extending from the central body and a free end. In some implementations, the first flexible arm and the second flexible arm may be removably attached to the central body. The flexible arms may be made of soft material or formed of thin pieces of material so that they can confirm to ear anatomy.

In some implementations, the first flexible arm and the second flexible arm may extend linearly from the central body when the device is in the inserted position. In some implementations, the first flexible arm may extend 180 degrees from the second flexible arm in the inserted configuration.

In some implementations, the first flexible arm may bend to conform to the concha of the ear when the device is in the rotated position. In some implementations, the second flexible arm may extend linearly from the central section in the rotated configuration.

In some implementations, the first flexible arm may bend to conform to the concha of the subject's ear in the rotated configuration when the device is inserted into the subject's right ear, and the second flexible arm may bend to conform to concha of the subject's ear in the rotated configuration when the device is inserted into the subject's left ear.

In some implementations, the device may comprise a first flexible hook-shaped arm and a second hook-shaped flexible arm. Each arm may have a connected end extending from the central body and a free end. When the device is in the rotated position, the free end of the first flexible hook-shaped arm may hook behind a superior portion of the subject's outer ear, and the free end of the second flexible hook-shaped arm may hook behind an inferior portion of the outer ear. In some implementations, the device may be symmetric around the major axis of the central body, such that the device may be used in the subject's left ear or right ear.

In some implementations, the first and second flexible arms or the first and second flexible hook-shaped arms may be configured to encourage the receptor into a position to obtain the diagnostic data from the ear drum when the device is in the rotated position.

In some implementations, the central body may comprise a patient-proximate outer surface, a distal outer surface opposite the patient-proximate outer surface, and an edge surface extending between the perimeters of the patient-proximate and distal outer surfaces. The patient-proximate and distal outer surfaces may be generally oval-shaped and comprise a major axis, a minor axis, and a first end and a second end located at opposite ends of the major axis. The extension is positioned near the first end of the patient-proximate outer surface. The extension may extend outward from the patient-proximate outer surface in a direction generally perpendicular to the patient-proximate outer surface. The connected ends of the first and second flexible arms may be positioned near the second end of the distal outer surface. The free ends of the first and second flexible arms may extend away from the central body in opposite directions that are parallel to the minor axis.

In some implementations, the device may be symmetric around the major axis of the central body, such that the device can be used in the subject's left ear or right ear. The connected ends of the first and second flexible arms may be positioned near the second end of the distal outer surface, and the free ends of the first and second flexible arms may extend away from the central body in opposite directions that are parallel to the minor axis. In some implementations, the device may be symmetric around the major axis of the central body, such that the device can be used in the subject's left ear or right ear. For example, the first flexible arm may bend to conform to the concha of the subject's ear in the rotated configuration when the device is inserted into the subject's right ear, and the second flexible arm may bend to conform to concha of the subject's ear in the rotated configuration when the device is inserted into the subject's left ear.

In some implementations, the extension may comprise a distal end connected to the patient-proximate outer surface and a patient-proximate tip extending into the subject's ear canal, the distal end being wider than the patient-proximate tip such that the extension is tapered to prevent over-insertion of the extension into the subject's ear canal.

In some implementations, the medical device may obtain diagnostic data from a subject's ear. The device may have an inserted position and a rotated position, as described above. The device may comprise a structure having a patient-proximate outer surface, a distal outer surface opposite the patient-proximate outer surface, and an edge surface extending between the perimeters of the patient-proximate and distal outer surfaces. Each of the patient-proximate and distal outer surfaces may have a generally rectangular shape with rounded corners and is defined by a major axis and a minor axis.

In some implementations, the structure may comprise a first portion of the edge surface located at one end of the major axis and a second portion of the edge surface at the opposite end of the major axis. The first portion may be located superior to the second portion in the inserted position. The first portion may be located inferior to the second portion in the rotated position.

In some implementations, in the inserted configuration, the device may be inserted into the ear without rotation. In some implementations, the device may have the rotated configuration after the device is inserted into the ear and is rotated approximately 180 degrees along the sagittal plane. In some implementations, the structure may be stabilized by the ear in the rotated configuration. In some implementations, in the rotated position, the first portion of the structure may be held in place by the subject's antitragus.

In some implementations, the device may comprise an extension extending from a central region of the patient-proximate surface of the structure. The extension may be sized and shaped for insertion into the ear canal and may extend at a non-perpendicular angle from the patient-proximate surface. In some implementations, the extension may be an attachment coupled to the structure. In some implementations, the extension may be integrally formed with the structure.

In some implementations, the extension may extend at an angle between 5 and 45 degrees from the horizontal plane. In some implementations, the extension may be positioned between the first portion and the second portion of the structure.

In some implementations, the extension may comprise a receptor to obtain the diagnostic data from the ear drum when the device is in the rotated position. The structure may be configured to encourage the receptor into a position to obtain the diagnostic data from the ear drum when the device is in the rotated position.

In some implementations, an area of the patient-proximate outer surface may be larger than a cross sectional area of the extension, such that the size of the structure prevents over-insertion of the extension into the ear canal.

In some implementations, the device may further comprise a first flexible arm and a second flexible arm, each arm having a connected end and a free end. In some implementations, the first flexible arm and the second flexible arm may extend linearly from the central body in the inserted configuration, and the first flexible arm bends to conform to the concha of the ear in the rotated configuration. In some implementations, the second flexible arm may extend linearly from the structure in the rotated configuration. In some implementations, the first flexible arm bends to conform to the concha of the ear in the rotated configuration when the device is inserted into the subject's right ear, and the second flexible arm may bend to conform to concha of the ear in the rotated configuration when the device is inserted into the subject's left ear. In some implementations, the first flexible arm may extend 180 degrees from the second flexible arm in the inserted configuration.

In some implementations, the extension may be positioned between the first portion and the second portion of the structure. The connected end of the first flexible arm may be positioned at the first portion, the connected end of the second flexible arms may be positioned at the second portion, and the free ends of the first and second flexible arms extend away from the structure in opposite directions that are parallel to the minor axis.

In some implementations, the first flexible arm and the second flexible arm may be removably attached to the structure.

In some implementations, the receptor may be an image capturing element configured to obtain visual data of the ear drum in the rotated configuration.

In some implementations, a system may image a subject's ear. The system may comprise a main body having a power source and an electronic element. The system may also comprise a device for obtaining diagnostic data from a subject's ear according to any of the implementations described herein. In some implementations, the device may be an earbud. The device may be connected to the main body.

In some implementations, the main body may be connected to the earbud via a wired connection. In some implementations, the main body may be wirelessly connected to the earbud over a communications link.

In some implementations, the main body may be configured to be placed over and behind the ear.

In some implementations, a non-medical profession may use the systems and methods described herein to perform an examination of a subject, as described in U.S. application Ser. No. 13/929,591, U.S. application Ser. No. 15/573,432, and U.S. Pat. No. 8,858,430 the contents of which are hereby incorporated by reference in their entirety. Performing the examination can include storing information about the subject's condition in a storage medium, include storage media accessed remotely (e.g., USB-accessible, LAN-accessible and/or internet accessible storage devices), as well as localized storage associated with the device (e.g., RAM or flash memory, SD cards, attached smartphone memory, etc.). The device may simultaneously and/or subsequently upload stored data to a general or specialized storage network, or remote access to the electronic storage medium can be provided over a computer or other communication network. The stored data may then be accessed by a competent medical professional or other caregiver, properly assessed, and recommendations regarding the condition and/or treatment can be transmitted or otherwise given to the subject or guardian. Various implementations disclosed herein include the manufacture, distribution and use of specialized and/or generalized devices that can be utilized by non-medical personnel (e.g., consumers) to collect subject information in a home-care or non-medical facility location.

In some implementations, the systems and devices described herein may be a part of and/or may be used in conjunction with electronic communications and/or display systems such as telephones, cell phones, smart phones, computers, wireless radios and/or other communications media known in the art. Desirably, the disclosed systems may allow a subject to transmit sufficient information over a communications link to the medical professional to enable assessment of the subject's condition, which may include information relevant to immediate and/or critical treatment of the condition that may not be readily apparent to the subject. In various implementations, the inventive systems may allow trained personnel to direct the subject's actions and/or use of the diagnostic device, such as requesting information regarding specific anatomical features which may be imaged by the diagnostic device in the hands of the subject as requested “real time” by the physician.

The link between the user and provider may be created using a variety of methods. A user may initiate a telehealth session by submitting a request for care. This request may be directed to a specific provider (for example, the subject's primary care physician), a limited network of providers, or it may be “crowd-sourced” to any available provider, which may facilitate a more rapid response. In addition, the request may be routed to a nurse hotline or call center that may provide a preliminary evaluation and as necessary forward the connection to an appropriate provider, such as a doctor or medical practitioner. A provider may then accept the request for care and, in various embodiments, a secure link between the user and the provider(s) can be established. This link may be as simple as a phone call but more desirably includes a video link between the user(s) and provider(s). Also, as described in the aforementioned, the secure link may only involve the transmission of information (e.g. video, voice and/or diagnostic information) and may not require “real time” live communication. The diagnostic information and/or other information such as a recorded voice and video transmission may be reviewed by a provider and/or software analysis tool offline from the user and a diagnosis or advice forwarded to the user.

### BRIEF DESCRIPTION OF THE DRAWINGS

FIG. 1A illustrates a representative diagram of a subject's outer ear:

FIG. 1B illustrates a representative diagram of a portion of a subject's inner ear;

FIG. 2 illustrates placement regions for the devices described herein:

FIG. 3 illustrates an exemplary device placement;

FIG. 4 illustrates exemplary boundaries of the auricle;

FIGS. 5-6 illustrates exemplary tissue that may be used to hold a device in place;

FIG. 7 illustrates exemplary forces exerted by ear tissue on devices:

FIG. 8 illustrates an exemplary device placed in an ear;

FIG. 9 illustrates exemplary forces exerted by the ear canal on a device:

FIGS. 10A-C illustrate an exemplary device in an inserted position and a rotated position, and a cross-section of the device;

FIGS. 11A-B illustrate an exemplary device with hooked flexible arms in an inserted position and a rotated position;

FIGS. 12A-B illustrate an exemplary device with hooked flexible arms in an inserted position and a rotated position;

FIGS. 13A-D illustrate a cross-sectional and front view of an exemplary angled device in an inserted position and a rotated position:

FIGS. 14A-B illustrate an exemplary angled device with flexible arms in an inserted position and a rotated position;

FIGS. 15A-B illustrates an exemplary device with retention arms in an unlocked position and a locked position;

FIGS. 16A-C illustrate an exemplary hinged device in an open position and a closed position, and positioned in an ear; and

FIG. 17 illustrates an exemplary device placed in an ear.

### DETAILED DESCRIPTION

To provide an overall understanding of the systems, method and devices described herein, certain illustrative embodiments will be described. Systems, methods and devices are described herein for obtaining visual data related to a subject's ear drum. The systems and devise described herein can be placed with little to no interference by anatomical features of the ear or near the ear. Such a system can enable an untrained layperson to safely obtain accurate visual information relating to the subject's ear, which may be transmitted to a physician or other medical professional for diagnosis of the subject's ear condition. Although the embodiments and features described herein are specifically described for use in connection with imaging a subjects inner ear, it will be understood that all the components and other features outlined below may be combined with one another in any suitable manner and may be adapted and applied to other types of devices relating to ears, including earphones for listening to audio signals, devices for measuring body temperature, devices for imaging the outer ear, and the like that require diagnostic information from the ear to tailor to the subject's needs. The configurations and positions described herein may allow devices to be placed in the ear with limited interference by the anatomy. For example, the stabilization and positioning elements described herein may be used to place an earbud such that a user can listen to music, or may be comprise an infrared thermometer to measure temperature of the eardrum and/or nearby tissue.

The systems, methods, and devices described herein may capture information and, in some implementations, send the captured information to a user's electronic device (e.g., a phone, tablet, computer, or any suitable device). In some implementations, the captured information is transmitted wirelessly. In some implementations, the captured information is transmitted over a wire. In some implementations, the captured information may sent to a medical profession for diagnosis. For example, the systems, methods, and devices described herein may be part of a telehealth system.

The systems and methods described herein may be constructed with one or more components or features which are structured for contacting parts of the body, creating an interface between the device and the body. These interfaces can make it easier to use a device or provide for improved safety, tolerance or comfort. For instance, interfaces may provide support or stability and reduce relative motion between components and the subject, help to encourage or achieve preferred positions and angles for accessing and capturing diagnostic information, reduce potential for injuring tissues, or provide soft and/or conforming interfaces for comfort. Interfaces may position a device with diagnostic elements in a preferred position or close to or within a window around preferred positions, thereby reducing the amount of user input or manipulation required to achieve a preferred position (a position capable of capturing desired diagnostic information). Interfaces may serve as locating features which help position a device at or close to a desired location or position prior to final positioning of other components or parts of the device, diagnostic elements, additional anatomic interfaces, or other accessing and capturing components. For example, an ear bud component with a through hole can be positioned in the ear prior to deploying an extension, attachment, or other part of the device through the ear bud into the ear canal. Similarly, an ear bud may be inserted in the ear and ear canal respectively, which may then be in a position to capture diagnostic information or may require additional manipulation, extension or other positioning.

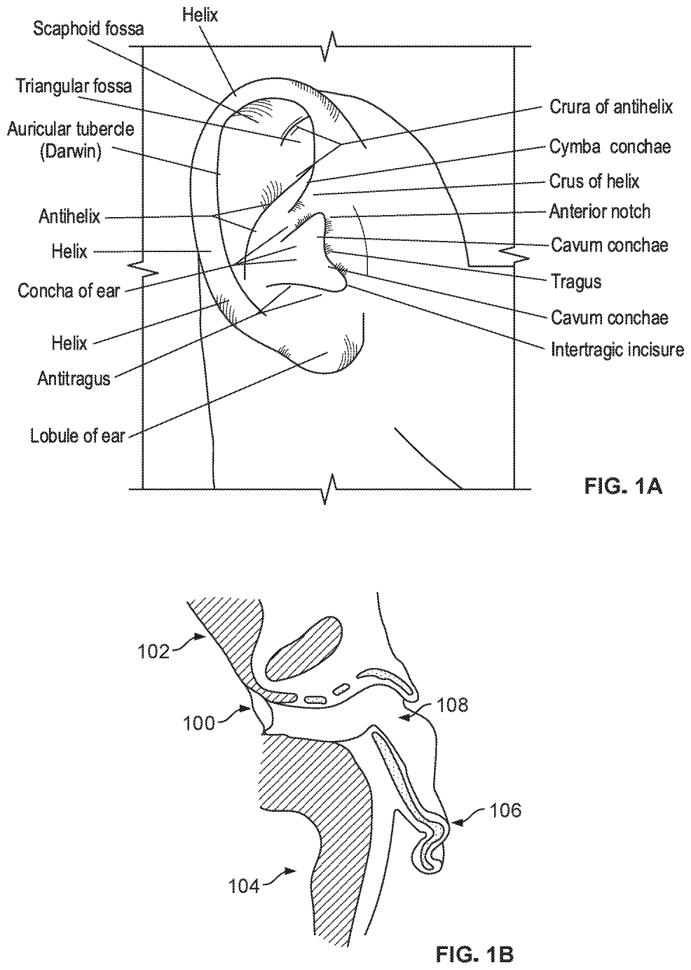

FIGS. 1A-B are provided to give context to the placement of devices described below. FIG. 1A shows a diagram of a subject's outer ear. FIG. A points to anatomical features of the outer ear including the helix, scaphoid fossa, triangular fossa, auricular tubercle (Darwin), antihelix, concha, tragus, antitragus, lobule, crura of the antihelix, cymba conchae, crus of helix, anterior notch, cavum conchae, and intertragic incisure. The outer ear consists of the visible portion of the ear, called the pinna or auricle, as well as the external acoustic meatus, or ear canal, which leads to the external surface of the tympanic membrane, or ear drum. The visible portion of the ear may also be referred to as the external ear. The concha is the bowl shaped part of the ear and leads into the ear canal. The tragus is located at the front of the concha and the antitragus is located below the concha. The antitragus is located behind as well as above the concha. The cavum concha is the inner portion of the concha that leads into the ear canal.

FIG. 1B shows a diagram of a portion of a subject's inner ear. Bones 102, 104 are on either side of ear canal 108. One end of ear canal 108 terminates at ear drum 100. The other end of ear canal 108 exits to the outer ear, partially defined by concha 106. The external acoustic meatus, or ear canal, is generally oval shaped and largest at the entrance. The ear canal is curved in an S shape, directed superiorly (up) and posteriorly (back) in the first section and then moving inferiorly (down) and anteriorly (forward). The canal is straighter in newborns, gradually taking on the general shape of an adult's ear canal through growth, and the ear canal is generally shaped similarly to an adult after 12 months of age. The canal entry is generally in the range of 9 mm vertically (inferior/superior direction) by 6.5 mm horizontally (anterior/posterior direction) for adults. The canal length varies from about 1.5 cm in infants to approximately 2.5 cm long for adults. The ear canal diameter then decreases to an average diameter of approximately 6-7 mm in adults and 3 mm in infants. The ear canal consists of a cartilaginous section and a bony section. The walls of the ear canal may be delicate and sensitive, especially when an ear infection is present. The bony portion of the ear canal, which runs the last ⅔ of the length of the ear canal, is especially delicate and sensitive and can be injured by objects such as the tip of a speculum. Tolerability problems can make it very difficult to view the ear drum and sometimes it is not possible at all. This situation is most common in younger children who are scared and uncooperative.

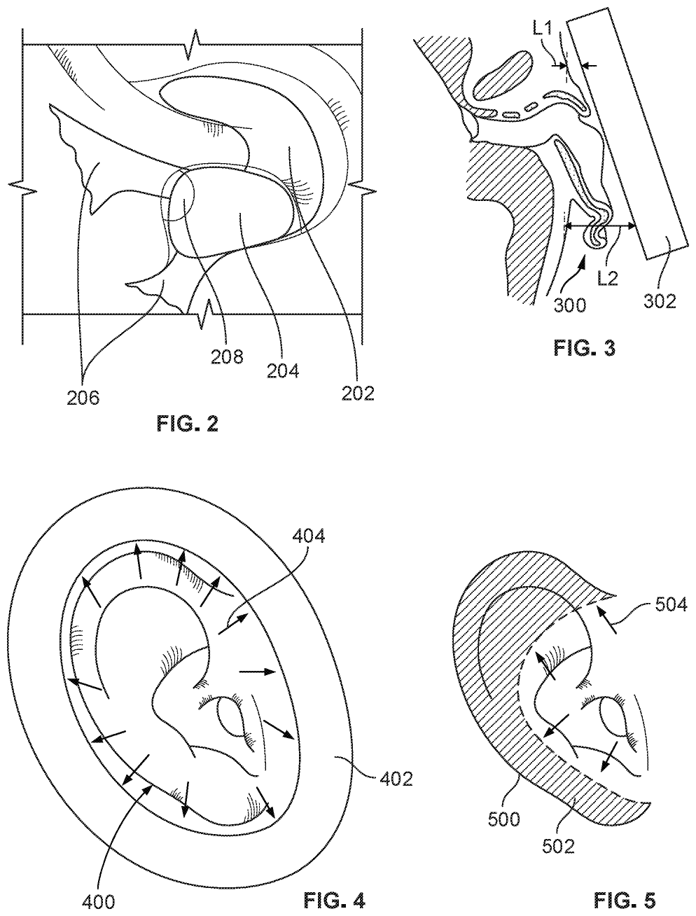

FIG. 2 shows placement regions for the devices described herein within a subject's ear. FIG. 2 illustrates three regions (first region 204, second region 202, and third region 206) within the boundary of the external visible ear (also referred to as the outer ear or auricle). First region 204 is the primary area where the systems and devices described herein are configured to be placed into the ear so that a portion of the system or device can extend into the ear canal. First region 204 and second region 208 are within the concha. First region 204 is within the cavum conchae, overlaps the entrance of ear canal 208, and is where a traditional earbud (e.g., for listening to music) may sit. First region 204 is bordered by the antitragus and the crus of the helix (as shown in FIG. 1A). Second region 202 curves in a superior and anterior manner from the cavum conchae into the cymba conchae. Second region 202 is another desirable area for the systems and devices described herein to be placed. In some implementations, parts of the device may be placed in region 202 are laterally offset to sections of the device placed in first region 204. In some implementations, sections of devices placed in second region 202 may be more flexible than sections placed in first region 204 to best fit the anatomy of the ear. Similarly, sections of a device extending into third region 206 may be laterally offset and/or more flexible than parts of the device placed in the second region 202 or first region 204 to better fit the anatomy. Third region 206 extends through the intertragic incisure and through the anterior notch. Device portions placed in third region 206 may also be more laterally offset and/or more flexible than parts of the device placed in second region 202 to better fit the anatomy of the ear. Devices portions located outside of these three regions 202, 204, 206 when a device is inserted are preferably offset more laterally and/or more are flexible than parts of the device intended for placement within these three regions 202, 204, 206.

FIG. 3 shows an illustrative device placement 302. Portions of devices placed in the placement regions 202, 204, 206 (described above in relation to FIG. 2) may be rigid or semi-rigid and may be contained within auricle 300. Parts of the device not placed in these regions should be flexible and/or configured to be placed lateral to the auricle as indicated by the device placement area 302. When a device is placed lateral to the auricle, it is at a non-parallel angle relative to the sagittal plane due to the ear anatomy, as evidenced by the placement area 302. The posterior end of placement area 302 is spaced a distance L2 away from the subject's head. The anterior end of placement area 302 is spaced a distance L1 away from the subject's head. In some implementations, L2 is greater than L1, such that placement area 302 is angled towards the subject's face in order to not interfere with auricle 300 during placement of a device in the location of placement area 302.

FIG. 4 shows illustrative boundaries of auricle 400. The boundaries of auricle 400 are indicated by the interior border of placement area 402. Portions of devices placed in the placement regions 202, 204, 206 (described above in relation to FIG. 2) may be rigid or semi-rigid and may be contained within auricle 400. Parts of the device not placed in these regions should be flexible and/or configured to be placed outside the boundaries of auricle 400, as shown by placement area 402. For example, an outer ring of a headphone device may be positioned in placement area 402. In some implementations forces represented by arrows 404 may exert an outward pressure on placement area 402.

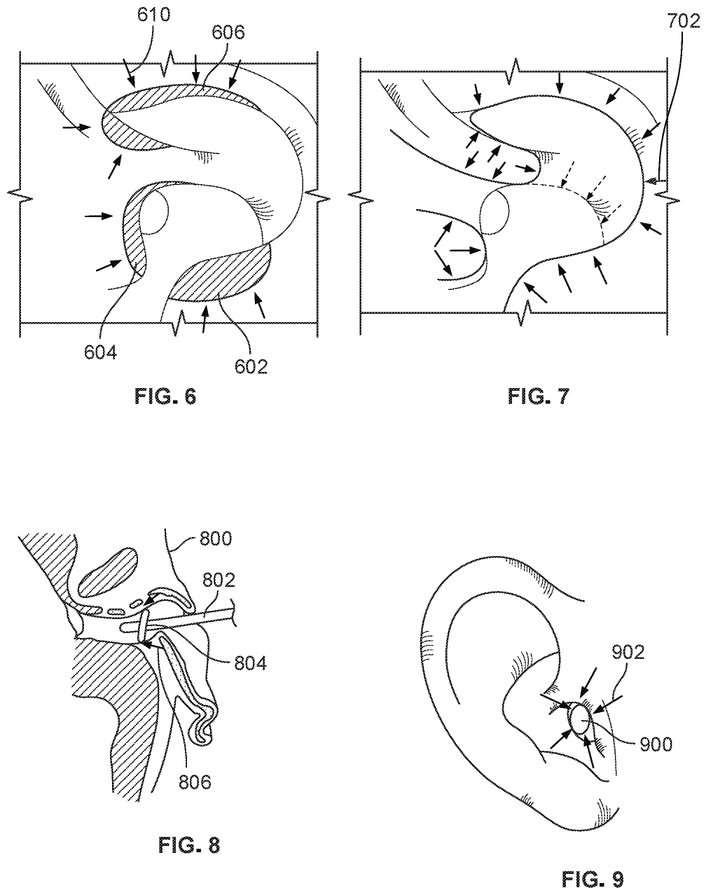

FIGS. 5-6 shows illustrative tissue that may be used to hold a device in place. Portions of the systems and devices described herein may be configured to be placed under or behind ear tissue, as shown by shaded region 502 in FIG. 5 and shaded regions 602, 604, 606 in FIG. 6. Shaded region 502 may comprise, for example, the helix, a portion of the scaphoid fossa, and a portion of the lobule of the ear. Shaded region 604 may comprise a portion of the tragus, shaded region 602 may comprise a portion of the antitragus, and shaded region 606 may comprise a portion of the antihelix and the crus of the helix.

When a device is placed behind any of shaded regions 502, 602, 604, 606, the ear tissue in those regions may exert a force on the device. Forces exerted by shaded region 502 are represented by arrows 504. For example, a device may hook behind the auricle 500, such that the device is supported by the outer portion of the auricle attached to and facing the subject's skull. Forces exerted by shaded regions 602, 604, 606 are represented by arrows 510. For example, a device may fit within the concha such that the device is held in place by shaded regions 602, 604, 606.

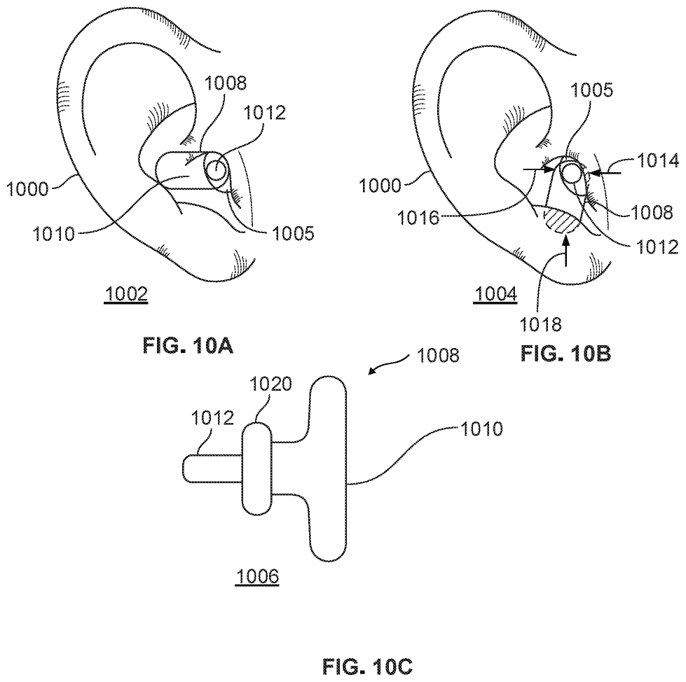

Parts of devices intended to fit near or behind shaded regions 502, 602, 604, 606 may be configured in many ways. Shaded regions 502, 602, 604, 606 show locations where tissue may exert medial, or inward, forces on device parts which serve as retention forces to help prevent the device pulling or falling out. For example, devices may be tapered, with parts of the device positioned just outside these regions (e.g., in a first inserted position). The position of the device may be adjusted (e.g., rotated) so that the device is fully seated into position. In the fully seated position, the device may fit snuggly into or taper out into these regions. In some implementations, the fully seated position is when the device is stably placed and positioned at least partially within the ear canal. In some implementations, adjusting the device into the fully seated position pushes the tissue out of the way, so that parts of the devices located in shaded regions 502, 602, 604, 606 are not behind or under tissue, but instead are pushing the tissue out of the way. In some implementations, devices may also be tapered in an opposite direction as previously stated. In some implementations, devices may have a small lip that snaps under one or more area of tissue (e.g., shaded regions 502, 602, 604, 606). As devices are pushed into tissue, the tissue is pressed inwards and then snaps back out as the lip of the device passes. Parts of such devices may be flexible. For example, the flexible portions of devices may be thin configurations, such as fingers or umbrella-like shapes, and/or may be made of soft/flexible material (e.g., silicone, other rubber, lower durometer urethanes, or any other suitable material). Parts of the device may be located outside shaded regions 502, 602, 604, 606 and then rotated into placed or otherwise translated or moved into these regions, as described below in relation to FIGS. 10-17.

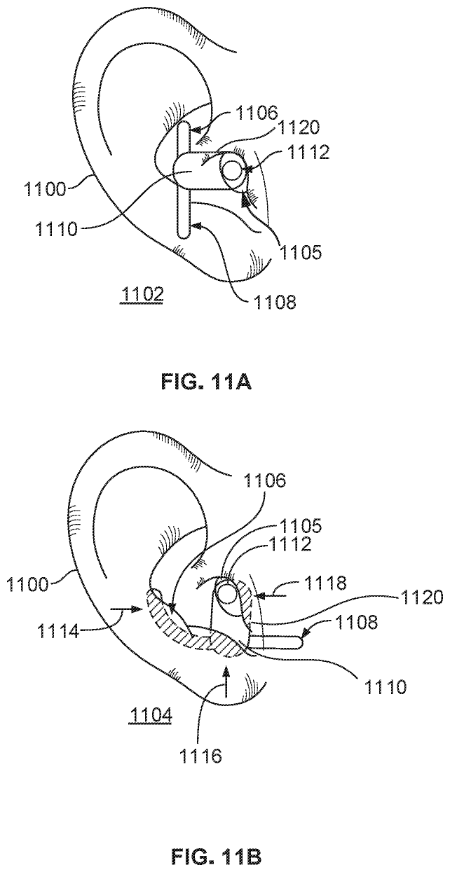

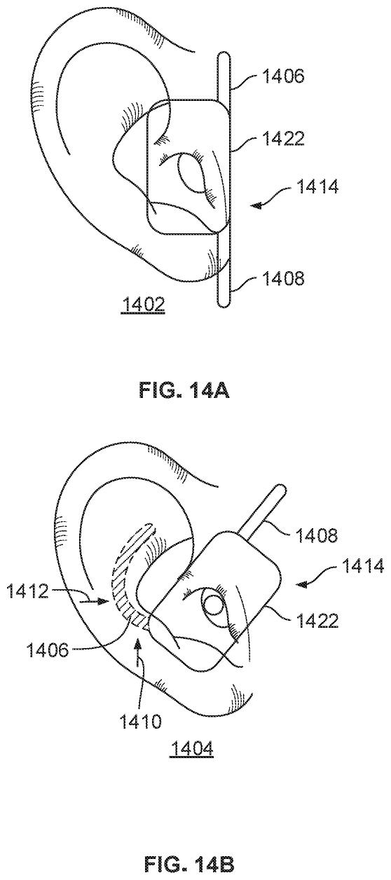

FIGS. 7-9 illustrate how tissue may exert forces (shown by arrows) on parts of devices located in different regions and locations. In some implementations, it may be preferable to have multiple forces acting on a device from different directions when the device is in a fully-seated or rotated position. For example, anterior and posterior forces (from front and back), inferior and superior forces (from below and above), as well as medial forces (forces acting inwards on device, or retention forces. Inward or retention forces (e.g., as shown by shaded regions 502, 602, 604, 606 in FIGS. 5 and 6 described above and as described below in relation to arms 1106 and 1406 of FIGS. 11 and 14, respectively) may also help to stabilize and hold the device in place, and may be created by friction forces acting on parts of devices located in other regions. As previously mentioned, devices may be flexible and snap into placed under tissue to help prevent device pullout. Devices may also be rotated into place. Ideally, devices are rotated at least 5 degrees to secure, and more preferably by at least 10 degrees, by at least 20 degrees, by at least 35 degrees, by at least 45 degrees, by at least 60 degrees, or by at least 90 degrees and up to about 120 degrees, up to about 150, up to about 180 or up to about 270 degrees, which may depend on specific device configurations, as described below in relation to FIGS. 10-17.

FIG. 7 shows the concha of an ear and shows forces represented by arrows 702 that can be exerted by the concha, tragus, antitragus, and antihelix, as well as any other suitable anatomical feature of the auricle on a device (e.g., device 1008 of FIG. 10) placed at least partially within the auricle. The forces represented by arrows 702 may hold the device in place or help to stabilize the device while the device captures data relating to the ear drum. FIG. 8 shows a device 802 with a stop 804 to prevent over-insertion. Forces represented by arrows 806 act against stop 804 to hold device 802 in the desired position, such that device 802 can acquire data relating to the ear drum. FIG. 9 shows an extension 900 within the ear canal. Extension 900 may be part of a larger device (e.g., device 802 of FIG. 8). The ear canal exerts forces represented by arrows 902 on extension 900 to hold the extension in a desired position.

FIG. 10A shows an illustrative device 1008 in an inserted position 1002, while FIG. 10B shows the device in a rotated position 1004, and FIG. 10C shows a cross-section 1006 of device 1020. Device 1008 comprises a central body 1010, an extension 1012, and a stop 1020 to prevent over-insertion of device 1008 into ear canal 1005. In both the inserted position 1002 and rotated position 1004, device 1008 sits within ear 1000. Central body 1010 is located within the concha of the ear 1000. In some implementations, central body may fit into a preferred placement region (e.g. first region 204 of FIG. 2). Central body 1010 comprises a patient-proximate outer surface, a distal outer surface opposite the patient-proximate outer surface, and an edge surface extending between the perimeters of the patient-proximate and distal outer surfaces. In some implementations, the patient-proximate and distal outer surfaces are generally oval-shaped and comprise a major axis, a minor axis, and a first end and a second end located at opposite ends of the major axis. Device 1108 can fit in both the right and left ears.

Extension 1012 fits into the ear canal 1005. Extension 1012 extends from the patient-proximate outer surface of central body 1010. In some implementations, extension 1012 extends perpendicularly from the patient-proximate outer surface. In some implementations, extension 1012 extends non-perpendicularly from the patient-proximate outer surface. For example, extension 1012 may extend at an angle of about 10 degrees, about 20 degrees, about 30 degrees, about 40 degrees, about 50 degrees, about 60 degrees, about 70 degrees, about 80 degrees, or any other suitable amount in any direction from the patient-proximate outer surface.

Device 1008 comprises a receptor. In some implementations, the receptor is located within central body 1010. In some implementations, the receptor is located within extension 1012. The receptor may be an image capturing element (e.g., a camera), an infrared sensor (e.g., a thermometer), or any suitable receptor. The receptor obtains diagnostic information relating to the ear drum.

In the inserted position 1002, device 1008 is inserted into the subject's outer ear, within the concha. Extension 1012 is inserted into ear canal 1005. In some implementations, device 1012 may be inserted into position 1002 directly, without any angling of device 1008. For example, the device 1008 may be inserted into the subject's outer ear without any manipulation of the outer ear. In some implementations, in the inserted position 1002, device 1008 may be inserted such that a major axis of device 1008 is approximately parallel to the transverse plane. In some implementations, in inserted position 1002, device 1008 may be inserted such that a major axis of device 1008 is approximately parallel to the sagittal plane. In some implementations, in position 1002, device 1008 is inserted at an angle. Stop 1020 prevents device 1020 from being over inserted into ear canal 1005.

Between inserted position 1002 and rotated position 1004, device 1008 is rotated in a counter-clockwise direction by approximately 90 degrees. The device is rotated to position device 1008 such that the receptor can acquire data relating to the ear drum. In some implementations, device 1008 may be rotated clockwise. In some implementations, device 1008 may be rotated greater than or less than 90 degrees. For example, device 1008 may be rotated about 60 degrees, about 70 degrees, about 80 degrees, about 100 degrees, about 110 degrees, about 120 degrees or any other suitable amount. In the rotated position 1004, portions 1014 and 1018 of central body 1010 are positioned under tissue. Preferred forces are created by ear tissue which help to hold device 1008 in place. The tragus exerts an anterior force on device 1008 (e.g., at portion 1014). The antitragus exerts an inferior force on device 1008 (e.g., at portion 1018). A posterior force from the back acts on the extension into the ear canal and a superior force or force from above is created by the weight of the device (e.g., at portion 1016). When the device is rotated to rotated position 104, the extension fits more snuggly into the ear canal 1005 than in inserted positon 1002. The rotation between inserted position 1002 and rotated position 1004 positions the receptor such that it can acquire data relating to the ear drum. For example, if an image capturing element is located within a tip of extension 1002, the rotation positions the tip of extension 1002 such that the image capturing element can take an image of the ear drum.

FIG. 11A shows an illustrative device 1120 with arms 1106, 1108 in an inserted position 1102, while FIG. 11B shows the device in a rotated position 1104. Device 1120 comprises a central body 1110 and an extension 1112. Central body 1110 is similar to central body 1010 and extension 1112 is similar to extension 1012, described above in relation to FIG. 10. Device 1120 is similar to device 1008 described above and comprises a receptor as described in relation to FIG. 10. First arm 1106 and flexible arm 1108 each have a connected end attached to central body 1110, and a free end extending away from central body 1110. In some implementations, the arms 1106, 1008 are made of flexible material. At least one advantage of flexible arms is that they bend around the ear tissue.

Device 1120 is inserted into ear 1100 in inserted position 1102. In inserted position 1102, first arm 1106 and second arm 1108 extend linearly from central body 1110. Arms 1106, 1108 lay approximately “on top” of the auricle 1100, such that they are not fully within the concha. Extension 1112 extends partially into ear canal 1105.

Between inserted position 1102 and rotated position 1104, device 1008 is rotated in a counter-clockwise direction by approximately 90 degrees. Device 1120 is rotated so that arm 1106 bends into the concha to help secure and stabilize the device. In rotated position 1104, arm 1108 extends through the intertragic incisure and exits ear 1100. Preferred forces are created by ear tissue which help to hold device 1020 in place. The tragus exerts an anterior force on device 1008 (e.g., represented by arrow 1118). The antitragus exerts an inferior force on device 1008 (e.g., represented by arrow 1116). A posterior force from the back acts on the extension into the ear canal and a superior force or force from above is created by the weight of the device. The concha exerts a posterior force on arm 1106 (e.g., represented by arrow 1114). Arm 1106 fits within the concha and, in some implementations, under the antihelix. Arm 1106 helps to hold device 1120 in a stable, proper position. Arms 1106, 1108 position central body 1110 such that extension 1112 extends at the correct angle for the receptor to acquire data relating to the ear drum.

In some implementations, device 1120 is configured for placement in either ear. For example, device 1120 may be symmetrical around a horizontal axis, such that arms 1106, 1108 are equal in length and are at an angle of approximately 180 degrees in inserted position 1102. In the right ear, arm 1106 may engage the concha (as shown), while in the left ear, arm 1108 may engage the concha (e.g., the opposite member engages the concha when placed in the opposite ear).

In some implementations, there may be just one arm (e.g., first arm 1106 or second arm 1108) extending from device 1120 and positioned towards the back or posteriorly on device 1120. In some implementations, device 1120 may also have a flexible joint or hinge that allows it to be rotated to fit the left or right ear.

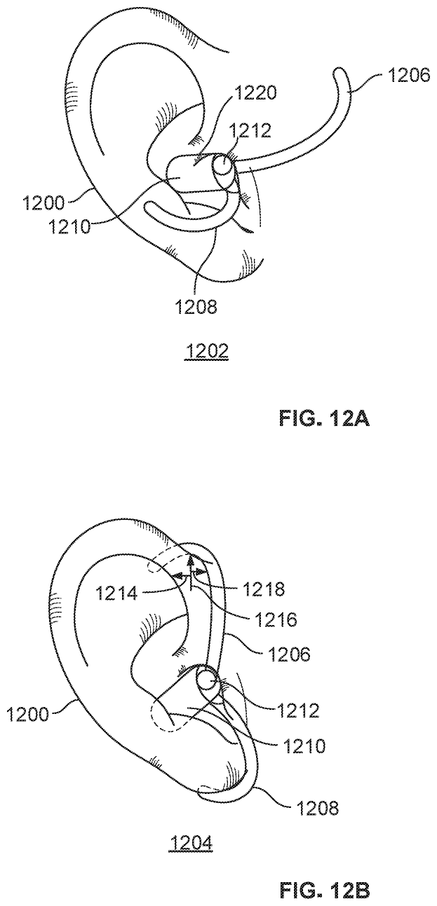

FIG. 12A shows an illustrative device 1220 with hooked flexible arms in an inserted position 1202 while FIG. 12B shows the device in a rotation position 1204. Device 1120 comprises a central body 1210 and an extension 1212. Central body 1210 is similar to central body 1010 and extension 1212 is similar to extension 1012, described above in relation to FIG. 11. Device 1120 is similar to device 1008 described above and comprises a receptor as described in relation to FIG. 10. First arm 1206 and flexible arm 1208 each have connected end connected to central body 1210. Arms 1206, 1208 are hook-shaped to interface with ear 1200. In some implementations, the arms 1206, 1208 are made of flexible material. At least one advantage of flexible arms is that they bend around the ear tissue.

Device 1220 is inserted into ear 1200 in inserted position 1202. In inserted position 1202, first arm 1206 and second arm 1208 extend from central body 1210. Arms 1206, 1208 lay approximately “on top” of the auricle 1200, such that they are not within the concha and do not yet hook around the auricle. Extension 1112 extends partially into ear canal 1105.

Between inserted position 1202 and rotated position 1204, device 1220 is rotated in a counter-clockwise direction by over 90 degrees and then allowed to “snap” back, by rotating in a slightly clockwise direction. This “over rotation” and snap back allows arm 1206 to hook over the top of the auricle 1200 and allows arm 1208 to hook beneath the auricle 1200. For example, arm 1206 may hook behind the helix while arm 1208 may hook behind the lobule of auricle 1200. The placement of arms 1206, 1208 helps secure and stabilize device 1208. Preferred tissue forces are created which help to hold device 1220 in place. Forces represented by arrows 1214, 1218, 1216 are exerted on arm 1206 and help to hold device 1220 in place. Arms 1206, 1208 position central body 1210 such that extension 1212 extends at the correct angle for the receptor to acquire data relating to the ear drum.

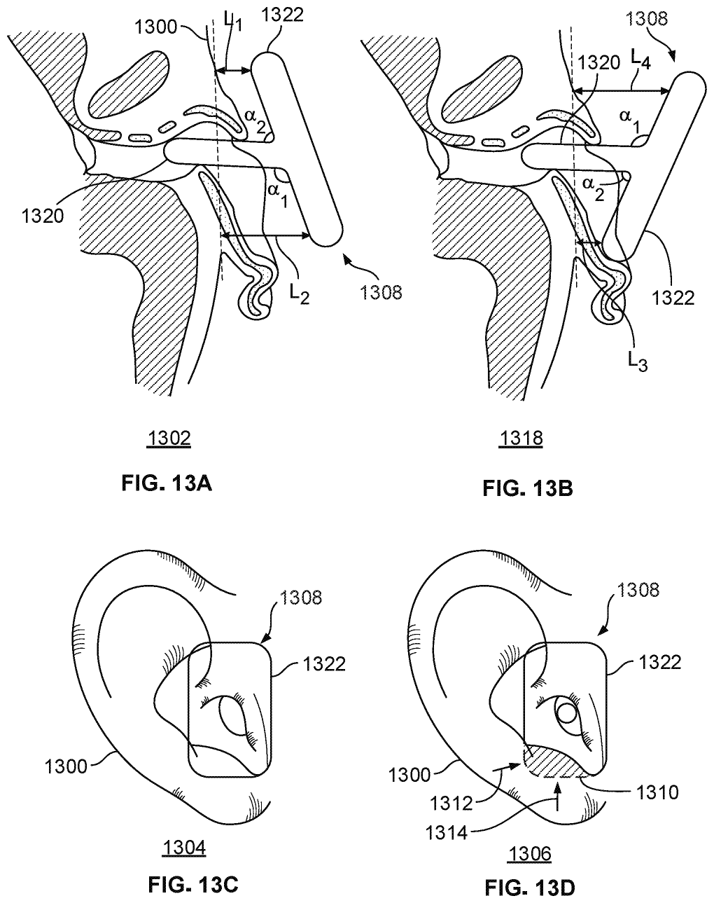

FIG. 13A and FIG. 13C show a cross-sectional and front view of an illustrative angled device 1308 in an inserted position, while FIG. 13B and FIG. 13D show device 1308 in a rotated position. The inserted position is shown in views 1302, 1304, while the rotated position is shown in views 1318, 1306. Device 1308 comprises central body 1322 and extension 1320. Device 1308 comprises a receptor. In some implementations, the receptor is located within central body 1322. In some implementations, the receptor is located within extension 1312. The receptor may be an image capturing element (e.g., a camera), an infrared sensor (e.g., a thermometer), or any suitable receptor. The receptor obtains diagnostic information relating to the ear drum.

Extension 1320 extends at a non-perpendicular angle from central body 1322, forming an acute angle α₁ and an obtuse angle α₂. Because of the angled extension 1320 and the ear anatomy, in inserted position 1302, 1304, the anterior part of device 1308 is initially positioned closer to the head (and ear canal entrance) as represented by distance L1 (formed from acute angle α₁) and the posterior part of device 1308 is further from the head as represented by distance L2 (formed from obtuse angle α₂), so the device does not interfere with auricle 1300 when initially placed.

Device 1308 is then rotated approximately 180 degrees counterclockwise into rotated position 1318, 1306. In the rotated position, the top anterior part of the device is now located more posteriorly (i.e., towards the back of the head). The top anterior part of device 1308 is thus positioned farther from the head (and ear canal entrance) as represented by distance L4 (formed from obtuse angle α₂), and the posterior part of device 1308 is farther from the head as represented by distance L3 (formed from acute angle α₁). This rotation positions the receptor such that it may acquire information from the ear drum.

As shown in rotated position 1306, a portion 1310 of device 1308 is positioned into the concha, helping to secure the device. Forces represented by arrows 1314, 1312 help to hold device 1308 in place. In some implementations, a second portion of device 1308 may be tucked under tissue in the concha (e.g., under the antihelix) or outside of the concha. In some implementations a central body similar to central body 1010 of FIG. 10 is secured to central body 1322 to better secure device 1308 within auricle 1300. In some implementations, device 1320 is configured for placement in either ear.

FIG. 14A shows an illustrative angled device 1414 with flexible arms 1406, 1408 in an inserted position 1402 while FIG. 14B shows the device in a rotated position 1404. Device 1414 comprises central body 1422 (similar to central body 1322 described above in relation to FIG. 13), an extension (similar to central body 1320 described above in relation to FIG. 13), and a receptor (similar to the receptor described in relation to FIG. 13). Additionally, device 1414 comprises a first arm 1406 and a second arm 1408. First arm 1406 and second arm 1408 each have connected end connected to central body 1422, and a free end extending away from central body 1422.

Device 1414 is inserted into the ear in inserted position 1402. In inserted position 1402, first arm 1406 and second arm 1408 extend linearly from central body 1422. Arms 1406, 1408 lay generally outside of the concha. The extension extends partially into the ear canal.

Between inserted position 1402 and rotated position 1404, device 1414 is rotated in a counter-clockwise direction between 45 and 180 degrees. Device 1414 is rotated so that arm 1406 bends into the concha to help secure and stabilize the device. In rotated position 1404, arm 1408 extends outside of the auricle. Preferred tissue forces are created which help to hold device 1414 in place. The concha exerts a posterior force on arm 1106 (e.g., represented by arrow 1412). Arm 1406 fits within the concha and, in some implementations, under the antihelix. Arm 1406 helps to hold device 1414 in a stable, proper position so that the receptor can obtain data from the ear drum.

In some implementations, device 1414 is configured for placement in either the left ear or the right ear. For example, device 1414 may be symmetrical around a horizontal axis, such that arms 1406, 1408 are equal in length and are at an angle of approximately 180 degrees from one another in inserted position 1402. In the right ear, arm 1406 may engage the concha (as shown), while in the left ear, arm 1408 may engage the concha (e.g., the opposite member engages the concha when placed in the opposite ear).

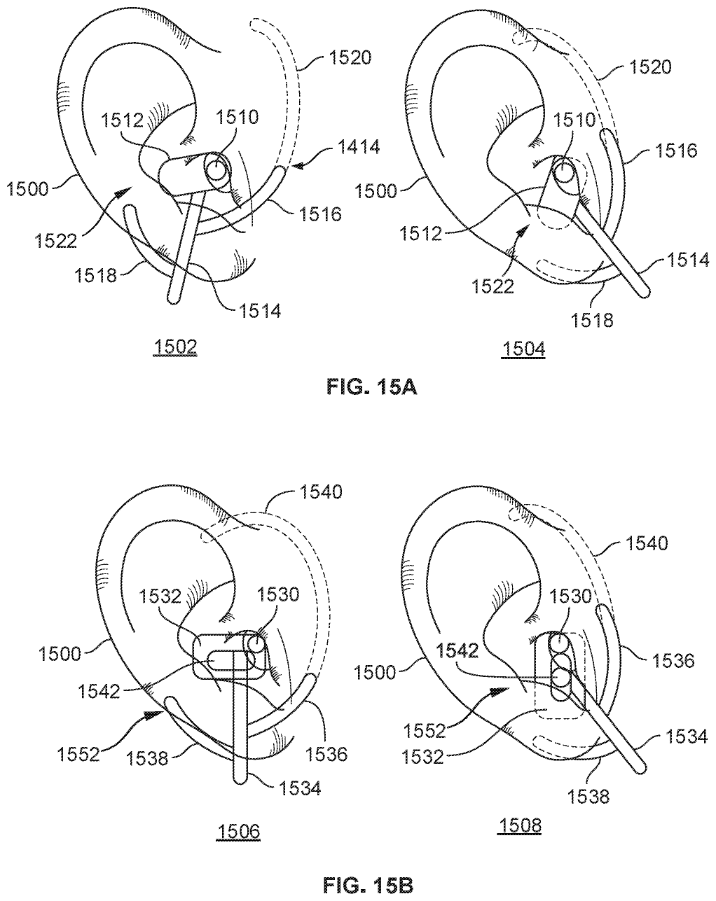

FIGS. 15A-B show two variations 1522, 1552 of a device with retention arms in an unlocked position 1502, 1506 and a locked position 1504, 1508. Device 1522 comprises central body 1512, extension 1510, central arm 1514, proximal side arm 1516, and distal side arm 1518. Central body 1510 comprises a patient-proximate outer surface, a distal outer surface opposite the patient-proximate outer surface, and an edge surface extending between the perimeters of the patient-proximate and distal outer surfaces. In some implementations, the patient-proximate and distal outer surfaces are generally oval-shaped and comprise a major axis, a minor axis, and a first end and a second end located at opposite ends of the major axis. In some implementations, central body 1512 is an earphone to deliver music to an ear. Proximal side arm 1516 and distal side arm 1518 each have a connected end connected to central 1514 and a free end extending away from central arm 1514 in opposite directions of one another. Central arm 1514 is linear and one end of central arm 1514 connects to central body 1512. Proximal side arm 1516 and distal side arm 1518 are curved. Optionally, proximal side arm 1516 may be an extended proximal side arm 1520 as shown.

Extension 1510 fits into the ear canal. Extension 1510 extends from the patient-proximate outer surface of central body 1512. In some implementations, extension 1510 extends perpendicularly from the patient-proximate outer surface. In some implementations, extension 1510 extends non-perpendicularly from the patient-proximate outer surface. For example, extension 1012 may extend at an angle of about 10 degrees, about 20 degrees, about degrees, about 40 degrees, about 50 degrees, about 60 degrees, about 70 degrees, about 80 degrees, or any other suitable amount in any direction from the patient-proximate outer surface.

In some implementations, device 1522 comprises a receptor. In some implementations, the receptor is located within central body 1512. In some implementations, the receptor is located within extension 1510. The receptor may be an image capturing element (e.g., a camera), an infrared sensor (e.g., a thermometer), or any suitable receptor. The receptor obtains diagnostic information relating to the ear drum.

In the unlocked position 1502, central arm 1514 extends in a generally inferior direction. The majority of central arm 1514, proximal side arm 1516, and distal side arm 1518 lay on top of or outside of ear 1500. In the locked position 1504, device 1522 is rotated in a counterclockwise manner from unlocked position 1502. In locked position 1504, central arm 1514 extends from central body 1512 out through or near the intertragic incisure. Device 1522 is rotated to allow distal side arm 1518 to lock into place under and in front of the inferior auricle. The weight of device 1522 and central arm 1514 prevent device 1522 from rotating further in the counterclockwise direction and “unlocking” (i.e., returning to unlocked state 1502). Proximal arm 1516 also prevents further rotation by engaging the outside of the tragus when rotated. In some implementations, extension 1520 extends over the top of auricle 1500 and hooks behind the ear, engaging the top of and/or behind the superior or upper portion of auricle 1500.

FIG. 15B is similar to FIG. 15A. Extension 1530 corresponds to extension 1510 of FIG. 15A, central body 1532 to 1512, device 1552 to 1522, central arm 1534 to 1514, proximal side arm 1536 to 1516, distal side arm 1538 to 1518, and proximal side arm extension 1540 to 1520. FIG. 15B differs from FIG. 15A, however, in that central arm 1534 is slidably connected to central body 1532. Central arm 1534 can slide within opening 1542 to allow central arm 1534 to lock into position between the unlocked state 1506 and locked state 1508. This slideable connection provides further stability and prevents device 1552 from over-rotating.

In some implementations, other means to prevent further rotation are provided. For example, central bodies 1522, 1552 may be “oversized” oval earbud that are restricted by the inferior tissue (antitragus) and/or tragus from over-rotating and being unlocked. In some implementations, a concha retention member is included. The concha retention member may extend off of central bodies 1522 or 1552.

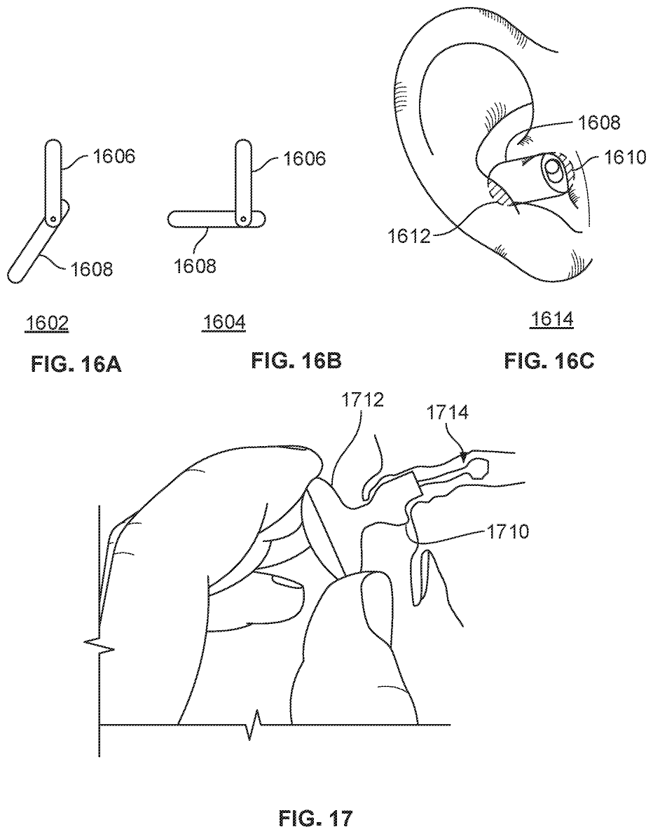

FIG. 16A shows an illustrative hinged device in an open position 1602, while FIG. 16B shows the device in a closed position 1604, and FIG. 16C shows the device in a closed position within an ear 1614. Extension 1606 is sized and shaped to enter the ear canal. Extension 1606 is connected by a hinge join to a central body 1608. Open position 1602 and closed position 1604 are views of the device from above, looking down into the page. The extension is placed into the ear canal with the device in open position 1602. The device is then “closed” by rotating central body 1608 into the concha and into closed position 1604. When the ear is pulled back, the tragus or other surrounding tissue pushes on part of the extension 1606 causing it to rotate and be directed more anteriorly, a more favorable position for imaging the ear drum when the ear is pulled back. The hinge between extension 1606 and central body 1608 allows the extension to better align with the ear canal in different positions and in different people with different shaped anatomy. Various types of joints and connections may be incorporated, including springs, ball joints, or any suitable connector. Flexible material may be used to form a hinge rather than using typical mechanical designs. This may allow motion in any direction and also be deflected by anatomy of different shapes or when the ear is pulled.

View 1614 shows the device in the closed position 1604 within an ear. When the device is in the closed position, a portion 1610 of central body 1606 is held in place by the tragus. Another portion 1612 of central body 1606 is held in place by the antitragus.

FIG. 17 shows an illustrative device placed in an ear, with a hand shown for scale. The device comprises a central body 1712, an extension 1714, and a stop 1710 to prevent over-insertion of the device. Extension 1714 includes bulb that houses the receptor described above. In some implementations, the receptor is a video chip with boroscope type lens design. Because of the bulb shape on extension 1714, the video chip may be of a greater size than could be accommodated in an extension without a bulb or ball joint. The device may also include any of the elements described above.

Any of the devices described herein may include a stop to prevent over-insertion of the device. In some implementations, the device comprises an extension sized and shaped to fit within the ear canal. The extension may comprise a first end connected to a central body and a second end configured to extend within the ear canal. The extension may, for example, taper from the first end to the second end, such that the width of the extension narrows as it extends into the ear canal. In some implementations, a stop is located between and connects the central body and the extension. The stop may have a wider width than the extension and may be configured to prevent over-insertion of the extension into the ear canal.

Any of the devices described herein may be part of a system including a main body housing electronics. The main body may include a power source (e.g., a battery). The main body may also include a processing element (e.g., a processor configured to process visual data) and a storage element (e.g., memory). In some implementations the device is connected to the main body by a wireless connection (e.g., via Wifi, Bluetooth, or any other suitable communications link). In some implementations, the device is connected to the main body by a wired connection (e.g., a detachable wire element running between the device and the main body).

In some implementations, the main body is placed over the ear (e.g., over and behind the auricle). In some implementations, a portion of the main body extends down to join to the device which includes an extension for insertion into the ear canal to image the ear drum. The extension may be adjustable (e.g., to fit different ages and ear sizes. Alternatively, the portion of the main body that extends down from the over-auricle section, may be flexible and/or stretchy, so the same portion can accommodate many ear sizes (e.g., because the portion can easily bend and curve if the full length is not required). This type of device may have a retaining feature (such as an “umbrella” feature, thin outward fingers, or a stop like stop 804 of FIG. 8) on the extension into the ear canal to help hold the extension in place.

Any of the devices described herein may include features to reduce the likelihood of unintentional destabilizing/unlocking (e.g., from the rotated position). These features may limit “anti-rotation” forces that could “dc-rotate” a device (e.g., from a rotated position to an inserted position) and/or release locking features. For example, locking features may include flexible arms (e.g., 1106, 1108 of FIG. 11), hook-shaped arms (e.g., 1206, 1208 of FIG. 12), flexible arms (e.g., 1406, 1408 of FIG. 14), arms (e.g., 1514, 1516, 1518, 1538, 1534, 1536 of FIG. 15), the hinge mechanism of (e.g., as shown in FIG. 16), or any other suitable feature. Forces that may unlock the device are most likely to occur during movement, such as activities like running or playing sports.

To reduce the likelihood of unintentionally destabilizing the device, the device may include a elements such as a dampening system (e.g., a weight on spring) and/or a gyroscope. The device may also include at least one accelerometer to detect motion and activate these elements. Additionally and/or alternatively, the device may be proportioned or a weight may be placed at a distance from the center of rotation of the device to create a moment. For example, the device may be of a rectangular shape with the major axis positioned vertically in an inserted position. When the device is adjusted to the rotated position, the major axis is moved at least slightly horizontally. This causes a weight at a distance from the center of rotation (e.g., the extension into the ear canal or an earbud earphone in the ear) and, therefore, creates a force or moment which will help prevent “un-rotation” and unlocking of the device.

Any of the extensions described herein (e.g., extension 1012 of FIG. 10, 1212 of FIG. 12, 1320 of FIGS. 13, 1510 and 1530 of FIG. 15, 1606 of FIG. 16, and 1714 of FIG. 17) may include bulb or ball joints, expandable tubes, light tubes, LED configurations, tapered speculums, Q-tip configurations and/or spring plunger extensions. In some implementations, a bulb or ball joint of the extension may house the receptor described above. For example, the joint may hold a video chip with boroscope type lens design. Because of the bulb shape on the extension, the video chip may be of a greater size than could be accommodated in an extension without a bulb or ball joint.

In some implementations, the extension comprises an expandable tip. The expandable tip may prevent wax blocking. In some implementations, the expandable tip includes a flexible nose cone. In some implementations, the expandable tip is a flexible entering tip configured to prevent damage to the ear canal. In some implementations, the expandable tip comprises wires that expand a tip, sheath, or extension that is pushed through a sheath.

In some implementations, the extension comprises light tubes and/or LED configurations. Micro “steps” in light tube at the tip of the extension may be configured to give an effective taper while still outputting light straight ahead. Light may be output to allow an image of the ear drum to be captured by a receptor.

In some implementations, the tip of the extension is in the shape in a miniature or tapered Q-tip. The tip may be tapered to mimic a speculum.

In some implementations, the extension is spring plunger extension. The device may be placed prior to placing an extension into the ear canal. For example, a device may be placed in an inserted position and then adjusted to a rotated position before the extension enters the ear canal. This allows a device with parts that are not in a preferred placement area, or otherwise configured to create some difficulty if placed with an extended tip or ear canal extension, to be maneuvered into place prior to extending the tip or extension into the ear canal. Once the device is placed within the auricle, the extension may be extended into place within the ear canal (e.g., via a spring system). This allows certain angles or shapes to be created in the ear canal extension that may have otherwise been difficult.

In some implementations, the systems and devices described herein can include variable size fittings to help secure the device into different size ears. The fitting may be part of existing elements of the device (e.g., an extension, stop, or flexible arm) or may be additional elements. In some implementations, variable fitting have various thicknesses to change the length of the exposed extension that enters the ear canal. In some implementations, the fittings can depress part of the extension back into the device (for example a spring loaded extension can be pressed back into the device) to vary the length of the extension. The length of the extension can also be modified without relying on fittings. For example, the length of the extension may be altered with a screw mechanism or gear mechanism (e.g., worm gear). In some implementations, the extension is tapered, or its diameter increased at a certain point, to ensure that an extension cannot be inadvertently over-inserted into a subject's ear. In this case, a larger diameter portion, for example located between 5 mm and 12 mm for a child, prevents entry into the ear canal and ensures safe operation.

In some implementations, the systems and devices described herein can be integrated with oral and other medical devices. For example, an ear imaging device or part of an ear imaging device may be inserted into a device intended for placement into an oral cavity for imaging the oral cavity and/or throat. The oral device may be configured as a clamshell and opened to receive the main device (e.g., the ear imaging device) and then closed prior to insertion into the oral cavity. The oral device may have a plastic shield near or at the distal tip (a “windshield”) that protects the camera and insides of the device from saliva and fog. This windshield may have anti-fog lubricant, channels may blow air or water across the “windshield” (similar to a windshield in a car). This oral device preferably has lights located outside the windshield so that light is not reflected by the windshield back at the camera. In this case, it is preferred that any lights on the main device, or ear imaging device, be turned off.

In some implementations, the systems and devices described herein may connect to additional diagnostic devices. For example, the device may comprise an additional component with wireless chip (e.g., Wifi, Bluetooth, etc.) that attaches to phone/computer/tablet, giving a wireless communications channel. The wireless communications channel may be for a diagnostic device such as an ear imaging device, a throat imaging device or a stethoscope device, and another communications channel for transfer of information to the cloud or other device or location and/or a video/voice call. The diagnostic device may also transfer information at a lower resolution than it captures information in order to maintain a live and steady stream of information (or to allow other information or uses of wireless channels). In this case, the device may store higher resolution information. In some implementations, the phone/computer/tablet may send information at a lower resolution to the cloud or other device or location. The information from any device can be later (or simultaneously) sent in higher resolution. The user or another person, such as a doctor, may select certain segments or specific snapshots of information to download/upload/send in higher resolution.

Specifically, in some implementations, the systems, methods, and devices described herein may be part of a telehealth system. A telehealth system will preferably include a method to remotely link one or more parties through communication devices and enable voice, video and/or text communication. Alternatively, a system may employ communication devices to allow a user to record and/or upload video, voice, text, background health information and/or diagnostic information, and enable a provider to evaluate and provide a diagnosis or advice without live communication with the user.

The communication component(s) may take a variety of forms. For example, the user may communicate with a computer, a tablet, a landline phone, a standard mobile phone, a smart phone such as the Apple iPhone, a unique communication device specialized for use with a telehealth system, or any other device that allows recording, transmission and/or uploading of voice, video, text, files and/or diagnostic information. In various implementations, the device will desirably allow receiving of similar information and enable the user to receive a diagnosis or advice from the provider. In one embodiment, the provider communication component is of similar design and capability. Although in other implementations, the user and provider can have dissimilar communications devices and components which still communicate and allow for sharing of text/voice/data as may be applicable.

The foregoing is merely illustrative of the principles of the disclosure and the apparatuses can be practiced by other than the described aspects, which are presented for purposes of illustration and not of limitation. It is to be understood that the apparatuses disclosed herein, while shown for use in percutaneous insertion of blood pumps, may be applied to apparatuses in other applications requiring hemostasis.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions and alterations are ascertainable by one skilled in the art and could be made without departing from the scope of the information disclosed herein. All references cited herein are incorporated by reference in their entirety and made part of this application.

1. A medical device for obtaining diagnostic data from a subject's ear, the device having an inserted position and a rotated position, the device comprising:
a central body;
an extension connected to the central body, the extension adapted for insertion into the ear canal;
a receptor located within at least one of the central body and the extension;
a first flexible arm and a second flexible arm, each having a connected end extending from the central body and a free end,
wherein the first flexible arm and the second flexible arm extend linearly from the central body when the device is in the inserted position, and the first flexible arm bends to conform to the concha of the ear when the device is in the rotated position,
the first and second flexible arms being configured to encourage a placement of the device such that the receptor is able to obtain the diagnostic data from the ear drum when the device is in the rotated position.

2. The device of claim 1, wherein the second flexible arm extends linearly from the central section in the rotated configuration.

3. The device of claim 1, wherein the first flexible arm bends to conform to the concha of the subject's ear in the rotated configuration when the device is inserted into the subject's right ear, and the second flexible arm bends to conform to concha of the subject's ear in the rotated configuration when the device is inserted into the subject's left ear.

4. The device of claim 1, wherein the extension is an attachment coupled to the central body.

5. The device of claim 1, wherein the extension is integrally formed with the central body.

6. The device of claim 1, wherein the first flexible arm and the second flexible arm are removably attached to the central body.

7. The device of claim 1, wherein the central body is stabilized by the ear in the rotated configuration.

8. The device of claim 7, wherein at least one of an inferior portion and a posterior portion of the central body is held in place by the antitragus and an anterior portion of the central body is held in place by the tragus in the rotated configuration.

9. The device of claim 1, wherein the first flexible arm extends 180 degrees from the second flexible arm in the inserted configuration.

10. The device of claim 1, wherein, in the inserted configuration, the device is inserted into the ear without rotation.

11. The device of claim 10, wherein the device has the rotated configuration after the device is inserted into the subject's ear and is rotated between 45 degrees and 180 degrees along a plane approximating the sagittal plane.

12. The device of claim 1, wherein the central body comprises a patient-proximate outer surface, a distal outer surface opposite the patient-proximate outer surface, and an edge surface extending between the perimeters of the patient-proximate and distal outer surfaces, wherein the patient-proximate and distal outer surfaces are generally oval-shaped and comprise a major axis, a minor axis, and a first end and a second end located at opposite ends of the major axis, wherein:
the extension is positioned near the first end of the patient-proximate outer surface, the extension extending outward from the patient-proximate outer surface in a direction generally perpendicular to the patient-proximate outer surface, and
the connected ends of the first and second flexible arms are positioned near the second end of the distal outer surface, and the free ends of the first and second flexible arms extend away from the central body in opposite directions that are parallel to the minor axis.

13. The device of claim 12, wherein the device is symmetric around the major axis of the central body, such that the device can be used in the subject's left ear or right ear.

14. The device of claim 12, wherein the extension comprises a distal end connected to the patient-proximate outer surface and a patient-proximate tip extending into the subject's ear canal, the distal end being wider than the patient-proximate tip such that the extension is tapered to prevent overinsertion of the extension into the subject's ear canal.

15. The device of claim 1, wherein the receptor is an image capturing element configured to obtain visual data of the ear drum in the rotated configuration.

16. A medical device for obtaining diagnostic data from a subject's ear, the device having an inserted position and a rotated position, the device comprising:
a central body;
an extension connected to the central body, the extension adapted for insertion into the ear canal;
a receptor located within at least one of the central body and the extension;
a first flexible hook-shaped arm and a second hook-shaped flexible arm, each arm having a connected end extending from the central body and a free end,
wherein, when the device is in the rotated position, the free end of the first flexible hook-shaped arm is hooked behind a superior portion of the subject's outer ear, and the free end of the second flexible hook-shaped arm hooks behind an inferior portion of the outer ear,
the first and second flexible arms being configured to encourage a placement of the device such that the receptor is able to obtain the diagnostic data from the ear drum when the device is in the rotated position.

17. The device of claim 16, wherein the central body is stabilized by the ear in the rotated configuration.

18. The device of claim 17, wherein an inferior portion of the central body is held in place by the antitragus and a superior portion of the central body is held in place by the tragus in the rotated configuration.

19. The device of claim 16, wherein the extension is an attachment coupled to the central body.

20. The device of claim 16, wherein the extension is integrally formed with the central body.

21. The device of claim 16, wherein the first flexible arm and the second flexible arm are removably attached to the central body.

22. The device of claim 16, wherein the first flexible arm extends 180 degrees from the second flexible arm in the inserted configuration.

23. The device of claim 16, wherein, in the inserted configuration, the device is inserted into the ear without rotation.

24. The device of claim 16, wherein the device has the rotated configuration after the device is inserted into the ear and is rotated between 45 degrees and 180 degrees along the sagittal plane.

25. The device of claim 16, wherein the central body comprises a patient-proximate outer surface, a distal outer surface opposite the patient-proximate outer surface, and an edge surface extending between the perimeters of the patient-proximate and distal outer surfaces, wherein the patient-proximate and distal outer surfaces are generally oval-shaped and comprise a major axis, a minor axis, and a first end and a second end located at opposite ends of the major axis, wherein:
the extension is positioned near the second end of the patient-proximate outer surface, the extension extending outward from the patient-proximate outer surface in a direction generally perpendicular to the patient-proximate outer surface, and
the connected ends of the first and second flexible arms are positioned near the second end of the distal outer surface, and extend away from the central body in opposite directions that are parallel to the minor axis.

26. The device of claim 25, wherein the extension comprises a distal end connected to the patient-proximate outer surface and a patient-proximate tip extending into the ear canal, the distal end being wider than the patient-proximate tip such that the extension is tapered to prevent over insertion of the extension into the ear canal.

27. The device of claim 25, wherein the device is symmetric around the major axis of the central body, such that the device can be used in the subject's left ear or right ear.

28. The device of claim 16, wherein the receptor is an image capturing element configured to obtain visual data of the ear drum in the rotated configuration.

29. A medical device for obtaining diagnostic data from a subject's ear, the device having an inserted position and a rotated position, the device comprising:
a structure having a patient-proximate outer surface, a distal outer surface opposite the patient-proximate outer surface, and an edge surface extending between the perimeters of the patient-proximate and distal outer surfaces, wherein each of the patient-proximate and distal outer surfaces have a generally rectangular shape with rounded corners and is defined by a major axis and a minor axis, the structure comprising a first portion of the edge surface located at one end of the major axis, a second portion of the edge surface at the opposite end of the major axis; and
an extension extending from a central region of the patient-proximate surface of the structure, the extension comprising a receptor to obtain the diagnostic data from the ear drum when the device is in the rotated position, the extension being sized and shaped for insertion into the ear canal and extending at a non-perpendicular angle from the patient-proximate surface, wherein:
the first portion is located superior to the second portion in the inserted position, and
the first portion is located inferior to the second portion in the rotated position,
the structure configured to encourage a placement of the device such that the receptor is able to obtain the diagnostic data from the ear drum when the device is in the rotated position.

30. The device of claim 29, wherein, in the rotated position, the first portion of the structure is held in place by the subject's antitragus.

31. The device of claim 29, wherein the structure is stabilized by the ear in the rotated configuration.

32. The device of claim 29, wherein, in the inserted configuration, the device is inserted into the ear without rotation.

33. The device of claim 29, wherein the extension is an attachment coupled to the structure.

34. The device of claim 29, wherein the extension is integrally formed with the structure.

35. The device of claim 29, wherein the device has the rotated configuration after the device is inserted into the ear and is rotated approximately 180 degrees along the sagittal plane.

36. The device of claim 29, wherein an area of the patient-proximate outer surface is larger than a cross sectional area of the extension, such that the size of the structure prevents over insertion of the extension into the ear canal.

37. The device of claim 29, wherein the extension extends at an angle between 5 and 45 degrees from the horizontal plane.

38. The device of claim 29, further comprising a first flexible arm and a second flexible arm, each arm having a connected end and a free end.

39. The device of claim 38, wherein the first flexible arm and the second flexible arm extend linearly from the central body in the inserted configuration, and the first flexible arm bends to conform to the concha of the ear in the rotated configuration.

40. The device of claim 38, wherein the second flexible arm extends linearly from the structure in the rotated configuration.

41. The device of claim 38, wherein the first flexible arm bends to conform to the concha of the ear in the rotated configuration when the device is inserted into the subject's right ear, and the second flexible arm bends to conform to concha of the ear in the rotated configuration when the device is inserted into the subject's left ear.

42. The device of claim 38, wherein the first flexible arm extends 180 degrees from the second flexible arm in the inserted configuration.

43. The device of claim 38, wherein the extension is positioned between the first portion and the second portion of the structure, and wherein the connected end of the first flexible arm is positioned at the first portion, the connected end of the second flexible arms is positioned at the second portion, and the free ends of the first and second flexible arms extend away from the structure in opposite directions that are parallel to the minor axis.

44. The device of claim 39, wherein the first flexible arm and the second flexible arm are removably attached to the structure.

45. The device of claim 29, wherein the receptor is an image capturing element configured to obtain visual data of the ear drum in the rotated configuration.

46. A system for imaging a subject's ear, the system comprising:
a main body comprising a power source and an electronic element;
an earbud according to any of claims 1, 16, and 29 connected to the main body.

47. The system of claim 46, wherein the main body is connected to the earbud via a wired connection.

48. The system of claim 46, wherein the main body is wirelessly connected to the earbud over a communications link.

49. The system of claim 46, wherein the main body is configured to be placed over and behind the ear.
